(19) United States
(12) Patent Application Publication
(10) Pub. No.: US 2025/0000693 A1
(43) Pub. Date: Jan. 2, 2025

(54) COMPOSITIONS, SYSTEMS AND METHODS FOR TREATING SUPRAPUBIC FAT

(71) Applicant: MIRAKI INNOVATION THINK TANK LLC, Cambridge, MA (US)

(72) Inventor: Emilia JAVORSKY, Watertown, MA (US)

(73) Assignee: MIRAKI INNOVATION THINK TANK LLC, Cambridge, MA (US)

(21) Appl. No.: 18/575,514

(22) PCT Filed: Jul. 5, 2022
(86) PCT No.: PCT/US2022/036110

§ 371 Date: Dec. 29, 2023

Related U.S. Application Data
(60) Provisional application No. 63/217,990, filed on Jul. 2, 2021.

(51) Int. Cl. A61F 7/12; A61F 7/00; A61K 9/08

(52) U.S. Cl. CPC A61F 7/12; A61K 9/08; A61F 2007/0022; A61F 2007/0048; A61F 2007/0063; A61F 2007/126

(57) ABSTRACT

Compositions, systems and methods are provided for the treatment of suprapubic fat, wherein the compositions comprise a cold solution comprising liquid water and/or ice particles, and optionally one or more additives and/or pharmacologic agents. The treatment of suprapubic fat includes cryolipolysis of the fat cells.

### TECHNICAL FIELD

This disclosure is directed to compositions, systems and methods for treating suprapubic fat.

### BACKGROUND

The male human body may comprise suprapubic fat where treating suprapubic fat may be performed to achieve penile enhancement and treat medical conditions. Current options for treating suprapubic fat are mainly limited to surgical interventions such as liposuction or surgical lipectomy, which is highly invasive and can be associated with undesirable side effects. Generally surgical treatments are not recommended for penile enhancement due to the risk of complications that can result in poor aesthetic and functional outcomes. Other treatments for non-surgical fat reduction such as laser and radio frequency are not suitable options for suprapubic fat reduction due to non-specific heating of the subcutaneous area.

Cryolipolysis, which employs fat-selective temperatures, is a promising alternative to selectively target and reduce suprapubic fat while also potentially improving skin laxity in the treatment area. However, conventional cryolipolysis is done with a topical applicator which greatly limits the precision of cooling, and carries the risk of cryoinjury to skin, which is especially relevant in the pubic area where skin is thinner relative to other areas of the body, and therefore may be more susceptible to cold injury. Similarly, the aggressive temperatures employed with topical cryolipolysis have been associated with nerve injury, making it high risk for this area.

Further, topical cryolipolysis has been associated with hernia risk due to the suction of the topical device in areas such as flank an abdomen. Given the risk of hernia formation, topical cryolipolysis employing suction would be high risk for the suprapubic area, given this is a main site of hernia formation. Accordingly, improved methods for treating suprapubic fat are desired.

### SUMMARY

The present invention provides compositions, systems and methods for treating suprapubic fat, by administering a cold solution to a subject in need thereof. The methods of the invention include selecting and targeting particular treatment site(s) in order to achieve the desired effects in the specifically desired location(s). Additionally, the methods of the invention allow quick, targeted administration of the cold solution, for example by injection, to the specifically chosen treatment site(s).

The present invention provides:

(1) A method for treating suprapubic fat, said method comprising administering an effective amount of a cold solution to a treatment site of a subject, wherein the treatment site comprises suprapubic fat, and wherein the cold solution comprises liquid water and/or solid ice particles.

(2) The method for treatment according to the above (1), wherein the cold solution comprises about 2% to about 70% solid ice particles, and optionally one or more additives.

(3) The method for treatment according to the above (1), wherein the cold solution comprises about 71% to about 100% solid ice particles, and optionally one or more additives.

(4) The method for treatment according to the above (1), wherein the cold solution is substantially liquid, and optionally comprises one or more additives.

(5) The method for treatment according to the above (1), wherein prior to administering an effective amount of the cold solution, imaging the treatment site of the subject to identify the suprapubic region.

(6) The method for treatment according to the above (1), wherein prior to administering an effective amount of the cold solution, physically and/or thermally isolating the treatment site.

(7) The method for treatment according to the above (1), wherein the cold solution is administered in a single treatment, or in a series of treatments.

(8) The method for treatment according to the above (1), wherein the cold solution is administered via a device selected from the group consisting of a needle, an expandable needle, a needle comprising more than one tip, a fenestrated needle, a fenestrated cannula, and an implant.

(9) A method for penile enhancement, said method comprising administering an effective amount of a cold solution to a treatment site of a subject in need thereof, wherein the treatment site comprises suprapubic fat, wherein the cold solution comprises liquid water and/or solid ice particles, and optionally one or more additives, and wherein the method results in a reduction in suprapubic fat.

(10) The method for treatment according to the above (9), wherein the cold solution comprises about 2% to about 70% solid ice particles, and optionally one or more additives.

(11) The method for treatment according to the above (9), wherein the cold solution comprises about 71% to about 100% solid ice particles, and optionally one or more additives.

(12) The method for treatment according to the above (9), wherein the cold solution is substantially liquid. and optionally comprises one or more additives.

(13) The method for treatment according to the above (9), wherein prior to administering an effective amount of a cold solution, imaging the treatment site of the subject to determine the presence of suprapubic fat.

(14) The method for treatment according to the above (9), wherein prior to administering an effective amount of the cold solution, physically and/or thermally isolating the treatment site.

(15) The method for treatment according to the above (9), wherein the cold solution is administered in a single treatment, or in a series of treatments.

(16) The method for treatment according to the above (9), wherein the cold solution is administered via a device selected from the group consisting of a needle, an expandable needle, a needle comprising more than one tip, a fenestrated needle, a fenestrated cannula, and an implant.

(17) A method for treating a medical condition, said method comprising administering an effective amount of a cold solution to a treatment site of a subject in need thereof, wherein the treatment site comprises suprapubic fat, wherein the cold solution comprises liquid water and/or solid ice particles, and optionally one or more additives, and wherein the method results in a reduction in suprapubic fat.

(18) The method for treatment according to the above (17), wherein the cold solution comprises about 2% to about 70% solid ice particles, and optionally one or more additives.

(19) The method for treatment according to the above (17), wherein the cold solution comprises about 71% to about 100% solid ice particles, and optionally one or more additives.

(20) The method for treatment according to the above (17), wherein the cold solution is substantially liquid, and optionally comprises one or more additives.

(21) The method for treatment according to the above (17), wherein prior to administering an effective amount of a cold solution, imaging the treatment site of the subject to determine the presence of suprapubic fat.

(22) The method for treatment according to the above (17), wherein prior to administering an effective amount of the cold solution, physically and/or thermally isolating the treatment site.

(23) The method for treatment according to the above (17), wherein the cold solution is administered in a single treatment, or in a series of treatments.

(24) The method for treatment according to the above (17), wherein the cold solution is administered via a device selected from the group consisting of a needle, an expandable needle, a needle comprising more than one tip, a fenestrated needle, a fenestrated cannula, and an implant.

(25) A composition comprising a cold solution, wherein:
the cold solution (1) comprises about 2% to about 70% solid ice particles, (2) comprises about 71% to about 100% solid ice particles, or (3) is substantially liquid;
the composition optionally comprises at least one additive; and optionally one or more suitable pharmacological agents; and
the composition is used to treat suprapubic fat in a subject.

(26) A method for treating suprapubic fat in a subject, said method comprising:
imaging one or more potential treatment sites in the subject to determine the presence of suprapubic fat,
thermally and/or physically isolating one or more potential treatment sites,
selecting one or more treatment sites which contain suprapubic fat, and
administering a cold solution into to the one or more selected treatment sites to treat the suprapubic fat of the subject,
wherein the cold solution comprises liquid water and/or ice particles, optionally one or more additives, and optionally one or more suitable pharmacological agents.

(27) A system for treating suprapubic fat, the system comprising:
a delivery device for delivering a cold solution comprising liquid water and/or ice particles, optionally one or more additives, and optionally one or more suitable pharmacological agents.

(28) The system according to the above (27), further comprising an imaging device.

(29) The system according to the above (27), further comprising an isolation device.

(30) The system according to the above (27), wherein the isolation device comprises a ring.

(31) The system according to the above (27), wherein the isolation device comprises a sleeve.

### BRIEF DESCRIPTION OF THE DRAWINGS

FIG. 1 illustrates locations of suprapubic fat in a human subject.

FIG. 2 illustrates an exemplary system to treat suprapubic fat.

### DETAILED DESCRIPTION

The invention provides compositions, systems and methods for treating

suprapubic fat. Treating suprapubic fat can include reducing suprapubic fat and/or improving suprapubic skin laxity. Treating suprapubic fat may result in penile enhancement where penile enhancement, including but not limited to, increased in perceived and functional penile length; improved sexual function and satisfaction; decreased risk of skin infection, treatment of lichen sclerosis, treatment of and/or decreased risk of skin degradation and skin necrosis, treatment of urinary dysfunction, and treatment of sexual dysfunction such as erectile dysfunction; improved aesthetic appearance of the lower abdomen/suprapubic region; and improvement in skin laxity of the suprapubic region. Additionally, treating suprapubic fat can treat medical conditions, including but not limited to, decreased penile length due to trauma, disease, skin contraction, and contracture post-penile prosthesis implantation; congenital conditions such as penoscrotal webbing and megaprepuce; and adult acquired buried penis common in individuals who are overweight or obese.

The methods of treatment according to the invention comprise administering a cold solution to a subject by any suitable method. The method of treatment will vary based upon the end result desired by the patient, and the patient's characteristics. The cold solution may comprise liquid water and/or ice particles. For example, the cold solution can be substantially liquid, substantially solid, or a slurry comprising both liquid and solid ice particles.

In some aspects, the cold solution may include water. In some aspects, the cold solution may include water and one or more additives. In some aspects, the one or more additives are inactive, biocompatible ingredients, including any substance (at or below their respective concentrations) in the FDA GRAS list, which is incorporated by reference in its entirety herein. In some aspects, the additives comprise one or more of a salt, a sugar, and a thickener.

In some aspects, the cold solution comprises potassium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises calcium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises sodium chloride at about 2.25% by mass or lower, for example at about 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass. In some aspects, the cold solution comprises magnesium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass.

In some aspects, the cold solution comprises sucrose at about 5% by mass or lower, for example at about 5, 4.5, 4, 3.5, 3, 2.5, 2, 1.5, 1, 0.5, or 0% by mass. In some aspects, the cold solution comprises dextrose at about 5.6% by mass or lower, for example at about 5.5, 5, 4.5, 4, 3.5, 3, 2.5, 2, 1.5, 1, 0.5, or 0% by mass. In some aspects, the cold solution comprises mannitol at about 4.95% by mass or lower, for example at about 4.5, 4, 3.5, 3, 2.5, 2, 1.5, 1, 0.5, or 0% by mass. In some aspects, the cold solution comprises lactose at about 0.45% by mass or lower, for example at about 0.4, 0.35, 0.3, 0.25, 0.2, 0.15, 0.1, 0.05, or 0% by mass. In some aspects, the cold solution comprises sorbitol at about 4.7% by mass or lower, for example at about 4.5, 4, 3.5, 3, 2.5, 2, 1.5, 1, 0.5, or 0% by mass. In some aspects, the cold solution comprises glycerol at about 2% by mass or lower, for example at about 1.9, 1.8, 1,7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass.

In some aspects, the cold solution comprises hetastarch at about 6% by mass or lower, for example at about 5.5, 5, 4.5, 4, 3.5, 3, 2.5, 2, 1.5, 1, 0.5, or 0% by mass. In some aspects, the cold solution comprises pectin at about 16.7% by mass or lower, for example at about 16, 15, 10, 9, 8, 7, 6, 5, 4, 3, 2, 1 or 0% by mass. In some aspects, the cold solution comprises polyethylene glycol at about 20% by mass or lower, for example at about 20, 15, 10, 9, 8, 7, 6, 5, 4, 3, 2, 1 or 0% by mass. In some aspects, the cold solution comprises gelatin at about 16% by mass or lower, for example at about 15, 10, 9, 8, 7. 6, 5, 4, 3, 2, 1 or 0% by mass. In some aspects, the cold solution comprises sodium methylcellulose at about 5% by mass or lower, for example at about 5, 4.5, 4, 3.5, 3, 2.5, 2, 1.5, 1, 0.5, or 0% by mass. In some aspects, the cold solution comprises a sodium alginate at about 5% by mass or lower, for example at about 5, 4.5, 4, 3.5, 3, 2.5, 2, 1.5, 1, 0.5, or 0% by mass. In some aspects, the cold solution comprises polyvinyl alcohol at about 5% by mass or lower, for example at about 5, 4.5, 4, 3.5, 3, 2.5, 2, 1.5, 1, 0.5, or 0% by mass. In some aspects, the cold solution comprises polyvinyl pyrrolidone (PVP) at about 5% by mass or lower, for example at about 5, 4.5, 4, 3.5, 3, 2.5, 2, 1.5, 1, 0.5, or 0% by mass. In some aspects, the cold solution comprises Xanthan Gum at about 0.75% by mass or lower, for example at about 0.7, 0.6, 0.5, 0.4, 0.3, 0.2, 0.1, 0.05 or 0% by mass. In some aspects, the cold solution comprises CMC at about 0.75% by mass or lower, for example at about 0.7, 0.6, 0.5, 0.4, 0.3, 0.2, 0.1, 0.05 or 0% by mass. In some aspects, the cold solution comprises guar gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises locust bean gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises gum tragacanth at about 1% by mass or lower, for example at about 0.9, 0.8, 0.7, 0.6, 0.5, 0.4, 0.3, 0.2, 0.1, 0.05 or 0% by mass. In some aspects, the cold solution comprises carbomer at about 1% by mass or lower, for example at about 0.9, 0.8, 0.7, 0.6, 0.5, 0.4, 0.3, 0.2, 0.1, 0.05 or 0% by mass.

Additional exemplary additives include bulking agents, such as sucrose, lactose, trehalose, mannitol, sorbitol, glucose, raffinose, glycine, histidine, PVP (K40); salts such as potassium, calcium, magnesium, hydrogen phosphate, hydrogen carbonate; buffering agents, such as sodium citrate, sodium phosphate, sodium hydroxide, tris base-65, tris acetate, tris HCl-65; tonicity modifiers, such as dextrose; collapse temperature modifiers such as dextran, ficoll, gelatin, and hydroxyethyl starch; antimicrobial preservatives such as benzalkonium chloride, benzethonium chloride, benzyl alcohol, chlorobutanol, m-cresol, myristyl gamma-picolinium chloride, paraben methyl, paraben propyl, phenol, 2-phenoxyethanol, phenyl mercuric nitrate, and thimerosal; chelating agents such as calcium disodium EDTA (ethylenediaminetetra acetic acid), disodium EDTA, calcium versetamide Na, calteridol, and DTPA; antioxidant and reducing agents such as acetone sodium bisulfate, argon, ascorbyl palmitate, ascorbate (sodium/acid), bisulfite sodium, butylated hydroxyl anisole, butylated hydroxyl toluene (BHT), 8roprano/cysteinateHCl, dithionite sodium, gentistic acid, gentistic acid ethanolamine, glutamate monosodium, glutathione, formaldehyde sulfoxylate sodium, metabisulfite potassium, metabisulfite sodium, methionine, monothioglycerol (thioglycerol), nitrogen, propyl gallate, sulfite sodium, tocopherol alpha, alpha tocopherol hydrogen succinate, thioglycolate sodium, thiourea, and anhydrous stannous chloride; solvents and co-solvents such as benzyl benzoate, oils, castor oil, cottonseed oil, N,N dimethylacetamide, ethanol, dehydrated ethanol, glycerin/glycerol, N-methyl-2-pyrrolidone, peanut oil, PEG, PEG 300, PEG 400, PEG 600, PEG 3350, PEG 4000, poppyseed oil, propylene glycol, safflower oil, sesame oil, soybean oil, vegetable oil, oleic acid, polyoxyethylene castor, sodium acetate-anhydrous, sodium carbonate-anhydrous, triethanolamine, and deoxycholate; buffers and pH-adjusting agents such as acetate, ammonium sulfate, ammonium hydroxide, arginine, aspartic acid, benzene sulfonic acid, benzoate sodium/acid, bicarbonate-sodium, boric acid/sodium, carbonate/sodium, carbon dioxide, citrate, diethanolamine, glucono delta lactone, glycine/glycine HCl, histidine/histidine HCl, hydrochloric acid, hydrobromic acid, lysine (L), maleic acid, meglumine, methanesulfonic acid, monoethanolamine, phosphate (acid, monobasic potassium, dibasic potassium, monobasic sodium, dibasic sodium and tribasic sodium), sodium hydroxide, succinate sodium/disodium, sulfuric acid, tartarate sodium/acid, and tromethamine (Tris); stabilizers such as aminoethyl sulfonic acid, asepsis sodium bicarbonate, L-cysteine, dietholamine, diethylenetriaminepentacetic acid, ferric chloride, albumin, hydrolyzed gelatin, 8roprano, and D,L-methionine; surfactants such as polyoxyethylene sorbitan monooleate (TWEEN® 80), Sorbitan monooleate, polyoxyethylene sorbitan monolaurate (TWEEN® 20), lecithin, polyoxyethylene-polyoxypropylene copolymers (PLURONICS®), polyoxyethylene monolaurate, phosphatidylcholines, glyceryl fatty acid esters, urea; complexing/dispersing agents such as cyclodextrins (e.g., hydroxypropyl-B-cyclodextrin, sulfobutylether-Bcyclodextrin); viscosity building agents such as celluloses such as sodium carboxymethylcellulose (CMC), hydroxyethylcellulose, hydroxypropylmethylcellulose, methylcellulose), acacia, gelatin, methyl cellulose, xanthan gum, polyethylene glycol, guar gum, locust bean gum, carrageenan, alginic acid, gelatin, carbopol, polyvinyl and pyrrolidone. Additives can be any of those found in Sougata Pramanick et al., “Excipient Selection in Parenteral Formulation Development,” 45 (3) Pharma Times 65-77 (2013), which is incorporated herein by reference in its entirety.

In some aspects, the cold solution can include one or more therapeutic agents, for example, an antioxidant, an anesthetic, a vasoconstrictor, an antibacterial, and a neuroprotectant.

The cold solution can be delivered to a subject such as a human or an animal, therefore the solution can be sterile and have an osmolality and pH such that it does not harm target or non-target tissue. In some aspects, the cold solution may have an osmolality of less than about 2,200 milli-Osmoles/kilogram. In some aspects, the cold solution may have an osmolality of less than about 1,000 milli-Osmoles/kilogram. In some aspects, the osmolality may be less than about 600 milli-Osmoles/kilogram. In some aspects, the pH is between about 4.5 and about 9.

In some aspects, the cold solution is substantially liquid such as the cold solution described in PCT/US2019/55605 filed on Oct. 10, 2019, which is incorporated by reference in its entirety herein. The cold solution can be cooled or supercooled to a temperature just before spontaneous nucleation occurs. Alternatively, the cold solution can be cooled or supercooled to a temperature approximate to or lower than where spontaneous nucleation occurs, then warmed such that all ice particles melt prior to delivery to a subject. One example of a cold solution is water that is supercooled. Water normally freezes at 273.15 K (0° C. or 32° F.), but it can be supercooled at standard pressure down to its crystal homogeneous nucleation at almost 224.8 K (−48.3° C./−55° F.). The supercooling process requires that water be pure and free of nucleation sites. This can be done by processes like reverse osmosis or chemical demineralization. Rapidly cooling of water at a rate on the order of 10^6 K/s avoids crystal nucleation and water becomes a glass, i.e., an amorphous (non-crystalline) solid. The temperature of the cold solution can be cooled to a temperature ranging from at about 10° C. to at about −50° C. One or more additives can be selected and included in the cold solution to change the freezing point of the cold solution.

In some aspects, the cold solution is substantially solid, i.e., substantially ice, such as the substantially solid solution. Substantially solid solutions, systems and methods for generating substantially solid solutions, and methods for administering substantially solid solutions are described in International Patent Application Ser. No. PCT/US2020/66093 filed on Dec. 18, 2020, which is incorporated by reference in its entirety herein. For example, the cold solution can comprise ice at a concentration of about 71% to about 100%, including 71, 75, 80, 85, 90, 91, 92, 93, 94, 95, 96, 97, 98, 99, 99.5, 99.9 and 100%. In some aspects, the cold solution may comprise 95 to 100% of ice in a solid state, e.g., an ice composition. In some aspects, the cold solution comprising 95% to 100% ice in a solid state is an ice needle composition, which may be generated and/or delivered via a cannula such as a needle.

In some aspects, the cold solution is a slurry, including a liquid and solid ice particles, such as the slurry described in International Patent Applications PCT/US2019/54828 filed on Oct. 4, 2019 and PCT/US2015/047301 filed on Aug. 27, 2015, both of which are incorporated by reference in their entirety herein. Systems and methods for making a slurry are described in International Patent Application PCT/US2019/55634 filed on Oct. 10, 2019, which is incorporated by reference in its entirety herein. One or more additives can be selected to optimize flowability which is the ability of the slurry to flow through a device or within a subject. For example, flowability describes how easy it is for the slurry to move, either within the system for making the slurry, a delivery device for delivering the slurry such as a cannula, or within the body of a human subject. Flowability is dependent on several factors, including ice particle size, ice particle shape (as they relate to the configuration of the delivery device, for example, needle gauge) and viscosity.

The slurry includes ice particles, for example at a concentration of about 2% to about 70%. In some aspects, the ice concentration is about 20% to about 50%, for example, at about 30% to about 40%, for example, about 31, 32, 33, 34, 35, 36, 37, 38, or 39%. The ice particles can be substantially rounded and uniform in shape and size. Ice crystal size can be based upon the size of the delivery device, for example, an ice particle size of about 100 μm may allow injection through a needle having an inside diameter of about 1.0 mm or smaller. In some aspects, the ice particle size may be less than about 1 mm, or less than about 0.25 mm. In some aspects, the temperature of the slurry can range from about −25° C. to about 10° C., for example, from about −6° C. to about 0° C., for example, about −5° C., −4° C., −3° C., −2° C., −1° C.

The type of cold solution(s) (i.e., substantially liquid, substantially solid, or slurry) and characteristics thereof (e.g., osmolality, volume, temperature, ice content, ice shape/size) can be selected based on the characteristics of the treatment site and desired outcome. A single treatment can include the delivery of one or more types of cold solution to one or more treatment sites via any suitable delivery method and any combination thereof.

The cold solution may comprise one or more pharmacologic agents. In some aspects of the invention, the pharmacological agent includes, but is not limited to PPAR agonists, including but not limited to PPARγ-agonists such as thiazolidinediones, (e.g., pioglitazone, rosiglitazone, lobeglitazone), NSAIDs (e.g., ibuprofen, salicylates, propionic acid derivatives, acetic acid derivatives, enolic acid derivatives, selective COX-2 inhibitors), indole, fibrate drugs (e.g., clofibrate, gemifibrozil, ciprofibrate, bezafibrate), aleglitazar, muraglitazar, tesaglitazar, saroglitazar, caffeine, genistein, isoproterenol, theophylline, cysteine, gallic acid, rutin and catechin.

In some aspects of the invention, the pharmacological agent includes exogenous agonists, including but not limited to BDNF, catecholamines (e.g., catechol, dopamine, norepinephrine, epinephrine, fenofibrate), IL-6, PTHrP, Meteorin-like (METR.NL), Irisin, Prostaglandins, VEGF, ANP/BNP, GDF5, FGF (FGF19, FGF21), BMPs (BMP4, BMP7, BMP8b). One or more exogeneous agonists may be incorporated into the cold solution.

In some aspects of the invention, the pharmacological agent includes drug agonists to promote endogenous signaling, including but not limited to BDNF, including but not limited to SSRI's (e.g., citalopram, escitalopram, fluoxetine, 1 1ropranolol, paroxetine, sertraline, dapoxetine) and SNRI's (e.g., atomoxetine, desvenlafaxine, duloxetine, levomilnacipram, milnacipran, sibutramine, tramadol, venlafaxine); catecholamines (e.g, amitriptyline, imipramine, nortriptyline, phenoxybenzamine, doxazosin, terazosin, prazosin, atenolol, metroprolol, 11ropranolol, labetolol, nifedipine, amlopdipine, diltiazem, verapamil, hydrazaline, isosorbide, minoxidil, ephedrine, pseudoephedrine, amphetamine, albuterol, caffeine, nicotine, theophyilline, levodopa, carbidopa); IL-6, including but not limited to antidepressants (e.g., venlafaxine, imipramine, serotonin, luoxetine); PTHrP, including but not limited to teriparatide, abaloparatide; Meteorin-like (METRNL): Irisin; prostaglandin analogues (e.g., travaprost, latanoprost, tafluprost, latanoprostene bunod, bimatoprost); VEGF (e.g., HIF1-alpha, PHD1, PHD2, PHD3, Azelnidipine, azilsartan, lercanidipine, nafcillin; ANP/BNP, including but not limited to sympathomimetics (methoxamine, midodrine, metaraminol, phenylephrine, amidephrine, clonidine, Dexmedetomidine, Fadolmidine, Guanfacine, Guanethidine, Xylazine, Tizanidine, Medetomidine, Methyldopa, Methylnorepinephrine, Norepin ephrine, Lofexidine, Medetomidine, Xylometazoline, Oxymetazoline, Cirazoline, Epinephrine, e rgotamine, etilefrine, indanidine, mephentermine, metaraminol, methoxamine, mivazerol, naphaz oline, norfenefrine, octopamine, phenylpropanolamine, propylhexedrine, rilmenidine, romifidine, synephrine, talipexole); GDF5; FGF (e.g., FGF19, FGF21, e.g., SIRT1 activators polyphenols such as reservatrol, methylene blue, metformin, NAD+); BMPs (e.g., BMP4, BMP7, BMP8b), including but not limited to apigein, disometin, isoliquirtigenin, and 4′-hydroxychalcone.

In some aspects of the invention, the pharmacological agent includes drug agonists, including but not limited to TGF-B, TNF-alpha and Retinoid Acid. In some aspects, the pharmacological agent includes β3 adrenergic receptor agonists, including but not limited to mirabegron, nebivolol and solabegron.

In some aspects of the invention, the pharmacological agent includes, but it not limited to, berberine, omega-3 fatty acids including alpha-linolenic acid, eicosapentaenoic acid, and docashexaenoic acid, melatonin, green tea extract, (−)-epigallocatechin-3-gallate (EGCG), menthol, ginsenoside, curcumin, artepillin C, bitter melon seed oil, butein, luteolin, farnesol, cryptotanshinone, albiflorin, trans-anethole, magnolol, xanthohumol, L-rhamnose, grape pomace extract, phytol, nobiletin, medicarpin, olaparib, dietary sea buckhorn pomace, zeaxanthin, trans-cinnamic acid, 6-gingerol, and apple polyphenols.

In some aspects of the invention, the pharmacological agent includes creatine.

In some aspects of the invention, the pharmacological agent includes stem cells, including but not limited to, mesenchymal stem cells, adipose-derived stem cells, human umbilical cord blood mononuclear cells, muscle-derived stem cells, urine-derived stem cells, neural crest stem cells, and endothelial progenitor cells.

In some aspects of the invention, the pharmacological agent includes stem cell or pericyte derived extracellular vesicles.

In some aspects of the invention, the pharmacological agent includes microRNAs.

In some aspects of the invention, the pharmacological agent includes platelet rich plasma and/or platelet derived biomaterials.

In some aspects of the invention, the pharmacological agent includes oxygen such as hyperbaric oxygen.

In some aspects of the invention, the pharmacological agent includes a vasodilator or short acting drug, including but not limited to, sildenafil, vardenafil, tadalafil, avanafil, papaverine, phentolamine, and alprostadil.

In some aspects of the invention, the pharmacological agent includes a stimulator of angiogenesis, including but not limited to, genes and proteins such as vascular endothelial growth factors such as VEGF-A or VEGF, PIGF, VGGF-B, VEGF-C, VEGF-D, and virus VEGF or VEGF-E; fibroblast growth factor family such as aFGF and bFGF; angiopoietin 1 (Ang-1); transforming growth factor-alpha/beta (TGF α/β); platelet-derived growth factor (PDGF); Hepatocyte growth factor/scatter factor (HGF/SF), Tumor necrosis factor-alpha (TNFα); Interleukin-1/8; angiogenin; ephrins; integrins (alpha v/beta 3, alpha v/beta 5, alpha 5/beta 1); cyclooxygenase-2 (COX-2); AAV-VEGF, PEI-VEGF, VEGF protein, Ad-Ang1+Ad-VEGF, Ad-COMP-Ang1, COMP-Ang1 protein, Ang4 protein, Ang4 protein, Apelin protein, bFGF hydrogel, Ad-adrenomedullin+Ad-Ang1.

In some aspects of the invention, the pharmacological agent includes a stimulator of nerve growth and/or regeneration, including but not limited to, genes and proteins such as AAV-BDNF; VEGF protein; HSV-NT3; HSV-GDNF; SHH protein (e.g., with nanofiber); GDF-5 protein; Ninjurin-1-mAb; TrkA-mAb; and GGF-2 protein.

In some aspects of the invention, the pharmacological agent includes an inhibitor of fibrosis, including but not limited to, genes and proteins such as Ad-Smad7 and TGF-β1 antagonist peptide.

In some aspects, the one or more pharmacologic agents may be encapsulated, for example, where the encapsulation comprises one or more of microencapsulation, nanoencapsulation, liposomal encapsulation and polymer encapsulation.

In some aspects, more than one pharmacological agent may be administered. In some aspects, the one or more pharmacological agents may be administered prior to, concurrently with, or subsequent to administration of the cold solution. When the one or more pharmacological agents are administered concurrently with the administration of the cold solution, the one or more pharmacological agents may be administered independently from the cold solution, or may be present in the cold solution.

In some aspects, the one or more pharmacological agents may be orally administered to the subject as a supplement to the administration of the cold solution. A subject may be treated by administration of the cold solution to treat suprapubic fat, resulting in the reduction of suprapubic fat and/or improvement in skin laxity, followed by administration of an oral supplement comprising one or more pharmacological agents, in order to maintain the treatment results.

Systems for treating suprapubic fat comprise any suitable means for administering a cold solution. In some aspects, the cold solution is administered via a delivery device such as cannula such a needle, an expandable needle, a needle comprising more than one tip, a fenestrated needle, a fenestrated cannula, and an implant. Exemplary delivery devices are described in U.S. Provisional Patent Application Ser. No. 63/062,680 filed on Aug. 7, 2020.

In some aspects, the cold solution may be administered by an implantable device, including but not limited to a balloon or a custom 3D-printed hollow implant. The balloon or hollow implant may be filled with the appropriate cold solution for the specific treatment and/or specific target site. The balloon or hollow implant may be re-filled one or more times for continuous treatment. The refilling of the balloon or hollow implant may occur on a pre-determined schedule, or may occur on an as-needed basis, such as after evaluation of prior treatments through monitoring and/or imaging. The cold solution to be administered via an implantable balloon or custom 3D-printed hollow implant may comprise one or more pharmacological agents, in order to augment the effects of the cold solution. The hollow implant may be in the shape of a tube, permitting inflow and outflow to allow circulation of the cold solution in and around the implant.

In some aspects, multiple hollow implants may be 3D-printed, then filled with the desired cold solution, to be administered over multiple treatment cycles. All of the implants may be filled at the start of the treatment, or each implant may be filled prior to implantation. Such a method may include printing multiple 3-D printed hollow implants, filling the hollow implant to be implanted with the desired cold solution, monitoring the treatment site for a reduction of suprapubic fat, removing the implant when the amount of suprapubic fat stabilizes, filling a second 3D-printed hollow implant with the desired cold solution, which may be the same or different from the previously implanted cold solution, monitoring the treatment site for a reduction in suprapubic fat, and removing the implant when the amount of suprapubic fat stabilizes. This method may be continued for multiple cycles, until the desired effect is achieved.

In some aspects, a cold solution, such as a substantially solid solution, can be administered to a treatment site, for example, via one or more incisions. In some aspects, the substantially solid solution can be generated using a mold where the mold can be 3D printed to represent the size and shape of suprapubic fat at the treatment site(s). In some aspects, a substantially solid solution can be administered in combination with an electrical current. For example, a substantially solid solution can be formed around the exterior of an electrical probe where the substantially solid solution and electrical current can be used to treat suprapubic fat.

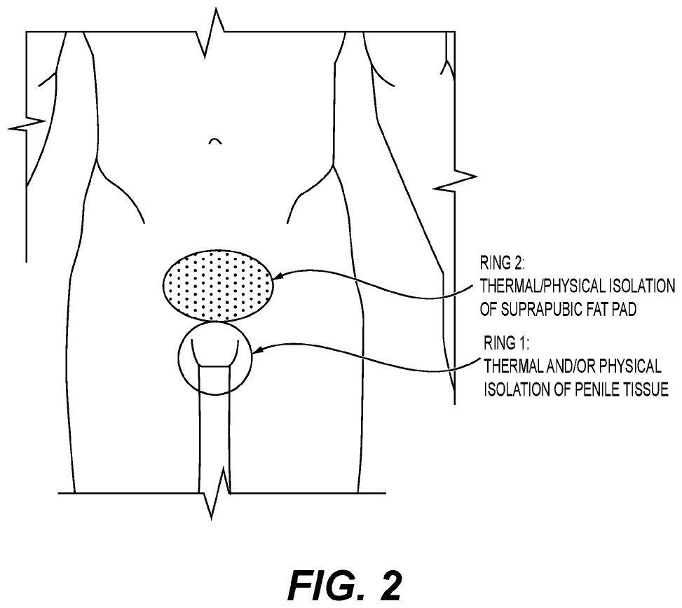

The system can further comprise one or more isolation devices configured to isolate, physically and/or thermally, enabling the suprapubic fat to be treated without affecting adjacent tissue; prevention of migration of the cold solution down the penile shaft; and active warming. The isolation device can comprise any suitable configuration, for example, a ring and/or a sleeve. In a nonlimiting example, as shown in FIG. 2, a first ring (Ring 1) can be configured to isolate the penile shaft from cold exposure and cold solution migration down the penile shaft, and a second ring (Ring 2) can be configured to isolate the suprapubic fat. One or more rings such as Ring 1 and Ring 2 can be used. In some aspects, Ring 1 and Ring 2 are configured as an integrated, single device or as separate components. In some aspects, the isolation device further comprises a strap to apply additional pressure to isolate a treatment site. In some aspects, the isolation device can be insulated and or expandable, e.g., inflatable, such that the isolation device actively warms the area.

The system can further comprise an imaging device where imaging may be utilized before, during, and/or after each treatment to form a baseline, monitor progress, and/or determine the efficacy of the treatment of suprapubic fat. The imaging device may be any suitable imaging device or procedure, including but not limited to an MRI, CT, ultrasound, PET, visual assessment, thermal imaging device, 3D imaging device, or any other device related to a known imaging procedure such as those described herein.

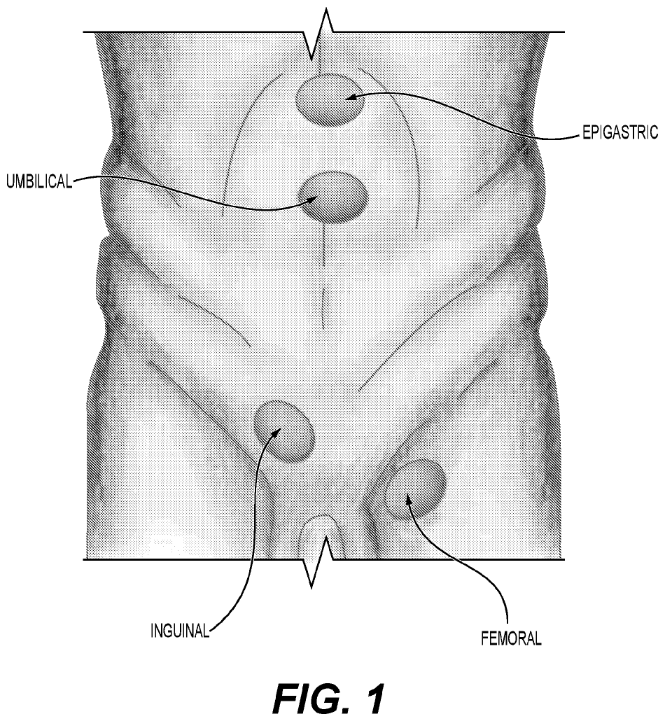

Methods of treating suprapubic fat comprise administering a cold solution to one or more treatment sites where the treatment sites are located in the suprapubic region, for example, as shown in FIG. 1. In some aspects, the method further comprises isolating the treatment site where isolating the treatment site can include physical and/or thermal isolation, enabling the suprapubic fat to be treated without affecting adjacent tissue; preventing migration of the cold solution down the penile shaft; and active warming. Isolation can be achieved by any suitable means, for example, via one or more isolation devices such as a ring and/or a sleeve. Imaging may be utilized before, during, and/or after each treatment to form a baseline, monitor progress, and/or determine the efficacy of the treatment of suprapubic fat. In some aspects, the method can include injecting image guidance to the suprapubic region to optimize treatment outcomes. The method can further comprise disrupting ligaments. For example, the ligaments can be disrupted mechanically via ice particles in the cold solution, ice needles, and/or the cold solution delivery device, e.g., a needle, or the ligaments can be disrupted thermally via a laser or radio frequency.

The cold solution may be administered in a single treatment, or may be administered as a series of treatments, for example a pre-treatment, followed by a treatment, followed by a post-treatment. In some aspects, the pre-treatment, treatment and post-treatment may occur in the same session. In other aspects, the pre-treatment and post-treatment may occur in sessions before and after the treatment session, respectively. In some aspects, the pre-treatment may occur 24 hours prior to the treatment. In another aspects, the pre-treatment may occur 24 hours after the treatment. In some aspects, there may be multiple pre-treatments, multiple treatments, and/or multiple post-treatments, which may occur in a single treatment session, or may be separated by one or more hours, one or more days, one or more weeks, or one or more months.

The cold solution may be administered continuously, such as through a catheter, providing consecutive or simultaneous effects. In another aspect, the cold solution may be administered periodically. In some aspects, imaging may be provided between periodic administrations in order to determine the effectiveness of the treatment, to determine the treatment site and/or to determine whether a new treatment site should be selected.

To reduce pain associated with injection, methods of the invention may further comprise administering an anesthetic to an area for treatment of the subject prior to injecting the cold solution, topically and/or via injection. For example, the anesthetic may be a local anesthetic, such as lidocaine. In certain aspects, the anesthetic may be administered to a subject a suitable amount of time in advance of the treatment in order to numb the injection area before treatment of the cold solution. In certain aspects, the cold solution may be topically applied to numb the injection site prior to injection.

Any suitable amount of cold solution that is safe for administering to the subject may be injected, based on subject characteristics, the treatment site and/or to produce desired effects of treatment. Potential treatment sites are visually demonstrated in FIG. 1, which shows known locations of suprapubic fat. A treatment may include delivering a volume of cold solution to one or more treatment sites. For example, when a cold solution is delivered via injection, the site may be treated via one or more injection sites, i.e., puncture site, and one or more deposition sites. The deposition site is where the cold solution is deposited, regardless of the injection site, and may be a different site than the injection site or the same site. One or more treatments can be required to achieve a desired effect.

In some aspects, the amount of cold solution injected may be about 2 L or less per injection site. In some examples, the amount of cold solution injected is about 1 mL to about 2 L per injection site. For example, different subjects have different amounts of suprapubic fat, and therefore, some subjects may require injection of greater amounts of cold solution in order to produce visible effects of reduction and removal of suprapubic fat. Other subjects may require multiple treatments to produce the desired effects.

In some aspects, the injection sites may form a pattern, such as a plow, fan, or grid-like pattern, or in a single bolus or multiple bolus injections. In some aspects, one injection site is used repeatedly, thereby reducing the number of injection sites and concomitant scarring potential. In a plow injection pattern, a single initial target injection site is used followed by a moving needle for additional deposition sites, for example in a linear pattern. In a fan injection pattern, deposition sites form an arc from 1 to 360 degrees. In a bolus injection, the cold solution is deposited in a single injection site.

The injection pattern and/or cold solution (including type and/or ingredients) can be determined based on the subject's profile, treatment plan (as described below), or based on the target site to be treated, i.e., treatment site. For example, an injection pattern and/or volume may be selected to optimize consistency of temperature at the treatment site. In an embodiment, the injection pattern and/or volume is selected in order to achieve gradient cooling of the tissue proximate to or at a treatment site or injection site. Injection techniques, including the patterns described herein, are known to those of skill in the art.

Treatment with the cold solution may be optimized for cosmetic or aesthetic results, for example to achieve smoothing and to avoid the appearance of sharp edges in the subcutaneous layer or layers. In some aspects, a profile can be created that correlates to the ice concentration, or ice coefficient, in the cold solution. The ice coefficient is defined as the percentage of ice, i.e., the percent by volume of water in a solid state in the cold solution, or the amount of ice by weight. For example, a cold solution with a higher ice coefficient can be used to treat the center of a treatment site, while a cold solution with a lower ice coefficient can be used to treat to the outer perimeter of the treatment site. Any of the cold solution properties such as ice coefficient, ice size and ice shape, can be varied to achieve a desired result.

In an aspect of the invention, a treatment plan may be created for a subject, for example to determine one or more of type of cold solution, the cold solution properties (for example, ingredients, tonicity and/or ice content, amount of cold solution to be delivered, delivery method(s) (e.g., ingestion, inhalation, injection, topical/contact, and/or incision), treatment sites such as suprapubic fat, a suitable device for administration of the cold solution, and whether to include one or more additional modalities in the subject's treatment. Factors considered in creating a treatment plan for a subject may comprise one or more of gender, height, body weight, body fat percentage, percentage of brown adipocytes, anatomy such as septae rigidity, lifestyle, vitals, medical history, lipid profiles, skin elasticity, medication, nutrition, supplements, demographic, vascularity of fat tissue, fat saturation, and the like. Fat saturation may be characterized by one or more of imaging, biopsy, and impedance measurement. In some aspects, after a plan is created for the subject, the amount of cold solution to be administered can be adjusted based on one or more of the area or areas to be treated, the areas to be treated, the depth of injection, and the injection pattern to be used, and which, if any, additional modalities are to be incorporated.

Imaging may also be utilized during creation of a treatment plan for a subject by collecting pre-, peri-, and/or post-injection data from one or more subjects. Information may be obtained through any suitable procedure, including but not limited to Magnetic Resonance Imaging (MRI), Computed Topography (CT), ultrasound, Positron Emission Tomography (PET), thermal imaging, Optical Coherence Tomography (OCT), and combinations thereof. Utilizing images of the target area, treatment site, surrounding areas, and/or other areas of interest, may provide detailed information regarding the most suitable treatment plan.

A computer or artificial intelligence system may be utilized to create a treatment plan and/or a post-procedure plan for a subject by collecting pre-, peri-, and/or post-injection data from multiple subjects. It is appreciated that the more data points, the more effective the artificial intelligence system will be in creating a treatment plan for a subject. For example, pre-, peri-, and/or post-injection data may be collected for each subject comprising one or more of gender, height, body weight, body fat percentage, the subject's anatomy, lifestyle, the subject's vitals, medical history, lipid profiles, skin elasticity, medication, nutrition, supplements, demographic, fat saturation, imaging data, treatment data and fat loss data. Data may be measured by any suitable means. For example, fat loss data may be measured by calipers or any imaging methods such as ultrasound, MRI, 3D photography, visual assessment, and the like. In some aspects, the system can be used to determine treatment eligibility, to order pre-treatment diagnostics/health screening, provide cost estimates, simulate results, provide information for subjects about the procedure, assist with patient intake, provide connectivity to service providers and Telehealth options, and provide an option for a subject to share individual treatment goals with a provider.

In some aspects, pre-procedure monitoring, for example to assist in creation of a treatment plan, and/or post-procedure monitoring, and/or a treatment modality can be performed via one or more monitoring devices including but not limited to a wearable physiological monitoring device, a sleep monitoring device, a metabolic monitoring device, a glucose monitoring device, monitoring of various biomarkers associated with health/disease such as markers associated with inflammatory and oxidative stress, blood work monitoring, hormone monitoring, body waste monitoring, white to brown fat conversion rate monitoring, mental well-being monitoring, taking physical measurements, e.g., using measuring tape, muscle mass measurement, 3D Image Scan, bioelectric impedance measuring, e.g., a scale, a handheld device, whole body measuring device, a direct segmental whole body composition measuring device, hydrostatic weighing centers, measurement of rate of healing of torn muscle fibers from exercise, indirect calorimetry, and a wearable to passively measure oxygen intake and carbon dioxide output, e.g., an RBC measurement, e.g., a device worn on a nostril.

Pre-or post-treatment steps may be utilized to optimize treatment results. For example, a massaging step may be utilized to increase fat cell damage and/or the mechanical force of the ice in the cold solution. In an aspect, the massaging is performed to puncture one or more cell membranes. The massaging step may be used to position or shape the cold solution post injection. Massaging can be performed by any mechanical means, for example by hand, vibration, an applicator, or by acoustic means. Imaging pre-injection can be utilized to create a treatment plan and may further be used to develop the profile for the subject. For example, the septae of the subject may be damaged prior to injection of the cold solution to allow the cold solution to flow more smoothly. In an embodiment, the septae in damaged by puncture. In another aspect, the septae is damaged by massaging.

Prior to, during, or after administration of the cold solution to the treatment site, visualization and identification of the target tissue, e.g., suprapubic fat, may be performed. In some aspects, visualization of the target tissue is performed using one or more known methods, including those previously described herein. The visualization step assists with identifying the location of the target tissue to which the cold solution will be administered, determining whether the target area should be adjusted, and/or monitors the efficacy of the treatment.

A pre-treatment and/or post-treatment step may include treatment utilizing a topically applied cold solution. A pre-treatment and/or post-treatment step may include implanting a balloon or a custom made 3-D printed hollow implant comprising a cold solution, wherein the balloon or implant may provide a continuous or variable administration of the cold solution. A pre-treatment and/or post-treatment step may include implanting a pharmacological device, which delivers a suitable pharmacological agent continuously, or at pre-determined intervals, including those previously described herein.

A pre-treatment step may include pre-activation of adipose tissue by activating local sympathetic input to fat using an energy-based device, including but not limited to a transcutaneous electrical nerve stimulation (TENS) device. A pre-treatment step may include pre-activation of adipose tissue through administration of a contrast agent and/or a pharmacological agent as disclosed previously herein. A pre-treatment step may include pre-activation through topical cooling, e.g., topical administration of the cold solution. A pre-treatment step may include pre-activation through topical administration of the cold solution, following by imaging through any known imaging method. The imaging method may be selected from the group of thermal imaging, CT, PET and MRI.

In some aspects, an incision is made to provide access to the target area, followed by administration of the cold solution, e.g., via injection, to the target area. The incision may also be used for imaging, in order to study and select the target area, and/or to monitor and evaluate the treatment efficacy. Such a treatment is helpful for difficult-to-reach adipose tissue.

In some aspects, the cold solution is administered to the subject via a port, which is implanted at or near the treatment site of the subject. The port may be utilized to administer the cold solution continuously, or at pre-determined intervals.

An implantable thermoelectric cooler may be implanted in a pre-treatment, treatment, and/or post-treatment step. A long-lasting drug eluting implant may be implanted, wherein the implant comprises a pharmacological agent selected from those previously described herein, in a pre-treatment, treatment and/or post-treatment step.

In some aspects, the administration of the cold slurry to a treatment site of a subject has additional benefits. For example, exposure to cold through administration of the cold solution alone, or in combination with other known methods, such as topical cooling of the skin, increases the metabolism of the subject, improves blood sugar of the subject and may result in weight loss.

In some aspects, a method of treatment comprises treatment of one or more layers of superficial adipose tissue, including superficial (sSAT) and deep (dSAT) layers of adipose tissue. The methods of treatment described herein may be used independently, in combination, or together with a treatment of superficial and/or deep adipose tissue, such as the methods described in International Application No. PCT/US2018/054834, filed Oct. 4, 2019, U.S. Provisional Application No. 62/953,272, filed Dec. 24, 2019, and International Application No. PCT/US2019/055605, filed Oct. 10, 2019, the contents of which are incorporated herein in their entirety.

In some aspects, prior to, during, or after a method of treatment according to the invention, the cold solution is administered to the subject in order to reduce inflammation, topically or via injection.

The methods of the invention may be utilized in combination with one or more additional modalities, as described in U.S. Provisional Application No. 63/035,139, filed Jun. 5, 2020, the contents of which are incorporated herein in their entirety. The one or more additional modalities may be administered in one or more of the pre-treatment, treatment or post-treatment sessions, or may occur before, between, or after one or more of the sessions.

The one or more additional modalities include, but are not limited to, energy, surgery, nutrition and/or wellness, exercise, and aesthetic, chemical and/or biological treatments. A modality which utilizes energy may include thermal energy, radiant energy, chemical energy, electrical energy and/or mechanical energy. In some aspects, the additional modality can be used to augment or supplement the treatment with cold solution.

In some aspects, thermal energy may be utilized to increase the temperature at or near the injection site and/or the treatment site. Thermal energy may be administered by any method known in the art, including but not limited to, hot or warm cloth, hot or warm water bottle, hot or warm bath, ultrasound, heating pad, heat therapy wraps, hydrocollator heat packs, and injection of a warm solution. The increase in temperature at or near the injection and/or treatment site may alter the physical characteristics of the tissue in and around the site, thus increasing fat reduction and improvement of the appearance of the skin. In some aspects, the tissue may be thinned and/or have increased flexibility. In some aspects, the blood flow at or near the site may increase, thus improving oxygenation and wound healing. In some aspects, the administration of thermal energy may reduce pain and/or inflammation at or near the site. In some aspects, the administration of thermal energy may activate a pharmacological agent which may be administered before, during, or after the administration of the cold solution.

In some aspects of the invention, radiant energy may be utilized through any device such as a laser and/or method known in the art, including but not limited to utilization of solar energy, visible light, infrared waves, radio waves such as radiofrequency, ultraviolet waves, X-rays, microwaves, and/or radium, in the treatment of the subject. In some aspects, a photosensitizer and light source can be administered to the treatment area, for example to improve the appearance of skin such as to reduce stretch marks. Examples of photosensitizers include but are not limited to 5-aminolevulinate, porphyrins, chlorins, bacteriochlorins, phthalocyanines, phenothiazinium salts, rose Bengal, squarines, BODIPY dyes, phenalenones, ruthenium compounds, rhodium compounds, hypericin, hypocrellin, riboflavin, and cur cumin. Examples of light sources include but are not limited to light-emitting diodes, lasers and intense pulsed light. In some instances, the radiant energy may be utilized to treat an area of the subject, or to diagnose and study an area of the subject.

In some aspects, chemical energy may be utilized through any device and/or method known in the art, in the treatment of the subject. In some aspects, chemical energy is administered by administering one or more substances to the treatment site, wherein the one or more substances cause an exothermic reaction thereby heating the treatment site. In some aspects, one or more substances can be administered to the treatment site where the one or more substances cause an exothermic reaction thereby heating the treatment site. In some aspects, when fat cells are subjected to cold, such as through the administration of a cold solution, the fat cells release energy as heat, thus further aiding in the reduction of fat.

In some aspects, electrical energy may be utilized through any device and/or method known in the art, including but not limited to electrical stimulation devices and electromagnetic devices, such as an electronic muscle stimulator or a transcutaneous electrical nerve stimulator (TENS), in the treatment of the subject. In some aspects, electrical energy may be administered in order to relax and/or tone muscles, increase blood circulation, manage pain, improve wound healing, and/or assist with drug delivery, for example, in the process of iontophoresis.

In some aspects of the invention, mechanical energy may be utilized through any device and/or method known in the art, in the treatment of the subject. In some aspects, mechanical energy may be administered through ultrasound, massage, vibration, pulsation and/or compression.

In some aspects of the invention, mechanical energy may be utilized through fractional wounding, wherein micro-holes are drilled in or around the treatment site, in order to stimulate collagen production and/or deliver the cold solution to the subject. A device containing a pre-fabricated array, for example, an array of needles or cannulas, may be utilized to drill the micro-holes in an appropriate pattern, wherein the pattern is in an appropriate shape for the size and shape of the administration site, and may include a grid, wherein the grid is a square-shape, a rectangular-shape, a circular-shape, or a triangular shape, a plow-shape, a fan-shape, a combination thereof, or a modification thereof.

In some aspects of the invention, mechanical energy may be utilized by administering filaments, wherein the filaments may be administered via a staple gun. In some aspects of the invention, the filaments are biodegradable, and may optionally be loaded with a pharmacological agent.

In some aspects of the invention, mechanical energy may be utilized by administering resorbable sutures in combination with the cold solution to the subject. In particular, a device is generated by placing one or more resorbable sutures in a mold, for example, a cannula, and placing a solution comprising water and optionally one or more additives in the mold, prior to cooling the mold and components therein. After achieving the desired ice coefficient through cooling, the cold solution further comprising one or more resorbable sutures is removed from the mold in an appropriate manner, and administered to the desired treatment site through an appropriate method, for example, via injection. After administration, the cold solution melts, through any suitable active or passive measure. The one or more resorbable sutures remain at or near the treatment site until resorption acting as an irritant to the tissue surrounding the treatment site. A cosmetic benefit occurs due to the increased collagen produced around the treatment site due to the presence of an unknown irritant, specifically the one or more resorbable sutures. The increased collagen promotes cell renewal, which provides cosmetically appealing results. In some aspects, the sutures may be provided in a pattern suitable to the treatment site.

In some aspects, mechanical energy may be utilized by administering ultrasound, for example via delivery device comprising a transducer. In some aspects, an ultrasound needle or catheter can be used to deliver energy, for example, the ultrasound catheter disclosed in Burdette, Everette et al., (2010), The ACUSITT Ultrasonic Ablator: The First Steerable Needle with an Integrated Interventional Tool, Proceedings of SPIE—The International Society for Optical Engineering, 7629. 10.1117/12.845972, the contents of which are incorporated by reference in its entirety.

In some aspects, mechanical energy may be utilized by administering shock wave therapy, for example, low intensity, extracorporeal shock wave therapy disclosed in Fojecki G L, Tiessen S, Osther P J S. Effect of Linear Low-Intensity Extracorporeal Shockwave Therapy for Erectile Dysfunction-12-Month Follow-Up of a Randomized, Double-Blinded, Sham-Controlled Study. Sex Med. 2018 March;6 (1): 1-7. doi: 10.1016/j.esxm.2017.09.002. Epub 2017 Dec. 21. PMID: 29275957; PMCID: PMC5815970, the contents of which are incorporated by reference in its entirety

A modality which utilizes surgery may include any appropriate surgical procedure known to those skilled in the art, including but not limited to an intervention to increase penile girth, such as a filler injection (including but not limited to fat, collagen, hyaluronic acid, and any compositions disclosed in U.S. Provisional Patent Application Ser. No. 63/001,889 filed on Mar. 30, 2020 which is incorporated herein by reference in its entirety), fat transfer; liposuction; implantation of a penile implant, for example an inflatable penile prosthesis. An intervention to increase penile girth via stimulation of subcutaneous fat, for example via a biologic, e.g., stem cell based, pharmacologic or device-based treatment.

A modality which utilizes nutrition and/or wellness may include monitoring and/or adjusting daily food intake, type of food and/or supplements, and/or calorie consumption of the subject to reduce fat. A nutrition and/or wellness modality may also include one or more of nutritional analysis, nutrition coaching, lifestyle coaching, weight loss, a personalized food plan/guide, personalized recipes, a personalized nutrition plan/guide, meal box service, grocery delivery, a farm/meat share subscription, regenerative medicine, traditional aesthetic medicine, mindfulness, sleep tracking, e.g., sleep cycle, assistance of a sleep coach, light-based smart-home technologies, e.g., hue-adjusted smart lights, smart-shades, integration with sleep data to optimize wake up and go to sleep timeframes, blue light management, obstructive sleep apnea therapy, indirect data metrics, including but not limited to meal purchase, use of refrigerator artificial intelligence, career planning and/or coaching, acupuncture, energy-based therapies, reiki, use of informational websites, use of smart technologies, e.g., Apple Health app, Apple Activity app, Apple fitness tracking, Calm app, Map My Walk app, Headspace app, MyFitnessPal, and Google Fit, use of a health cloud, e.g., Sales Force 360 CRM, spiritual guidance, participation in faith based/spiritual communities, participation in faith based/spiritual experiences, financial planning, retirement planning, finance tracking, e.g., earnings and spending, and travel planning.

A modality which utilizes exercise may include implementing, or adding to, an exercise regimen to reduce fat, increase muscle and/or maintain weight and muscle content. An exercise modality may also include one or more of personal training or coaching, physical therapy, at home workout, muscle stimulation, supplements, e.g., personalized supplements, meditation, yoga and tracking performance metrics.

A modality which utilizes self-optimization may include one or more of cosmetics including but not limited to hair products, skin products and nail products; aesthetic medicine approaches; a skin care regimen or treatment including but not limited to skin typing, personalized skin care regimen, topicals, skin tightening, cryangiogenesis, wrinkle management, treatment of dark spots, treatment of hyperpigmentation, texture, hydration, environmental stressor protection, e.g., sun care, pollution prevention, treatment of non-facial skin including but not limited to scars, striae, cellulite, skin laxity, keratosis pilaris, hyperhidrosis, folliculitis, and intertrigo; and a cosmetic procedure such as a non-invasive, minimally invasive or invasive procedure.

A chemical and/or biological modality may include treatment of the subject using small molecules, large molecules, mid-size molecules, protein degraders, antibody drug conjugates, gene therapy and/or molecular probes. Such a treatment may include the administration of one or more a pharmacological agent which may augment the treatment of suprapubic fat. The administration of the pharmacological agent may be in any suitable method. In some aspects of the invention, the pharmacological agent includes, but is not limited to the pharmacological agents previously disclosed herein. In some aspects of the invention, the one or more pharmacological agents may be administered prior to, concurrently with, or subsequent to administration of the cold solution. When the one or more pharmacological agents are administered concurrently with the administration of the cold solution, the one or more pharmacological agents may be administered independently from the cold solution, or may be present in the cold solution.

A modality that augments or supplements the treatment using a cold solution, e.g., one or more injections of cold solution, can enhance the cold solution treatment, reduce side effects of the cold solution treatment and/or improve the outcome of the cold solution treatment. Modalities to enhance the cold solution treatment include but are not limited to administering an anesthetic prior to the cold slurry treatment; a modality to prevent leakage of the melted slurry, e.g., a dressing such as a superabsorbent polymer/water-absorbing polymer dressing such as a hydrogel dressing, gauze dressing, alginate dressing, hydrofiber dressing, foam dressing, medical bandage or adhesive; a modality to keep the subject warm before, during or after the treatment, e.g. a warming blanket, a non-contact thermal light source such as an infrared mask, hot water bottle, heating pad, heated treatment table, and a mild capsaicin cream (to increase blood flow and thus the perception of warmth); and a modality to minimize any noise created by the device during treatment, e.g., ear plugs, noise cancelling headphones or a wearable such as a foam padded hat. Modalities to reduce side effects include but are not limited to modalities to decrease bruising and/or inflammation caused by an injection, e.g., a topical comprising arnica and/or menthol: and a modality to improve healing and/or reduce scarring at an injection site, e.g., a topical such as a retinoid, corticosteroid cream, onion extract cream, petrolatum ointment, a dressing such as a silicone dressing, and a mechanical modality such as a massage or vibration device to relieve tension at the injection site. Modalities to improve outcomes include but are not limited to a modality to break up the fibrous septae in advance of a treatment, e.g., a mechanical or thermal device as described herein; a modality to heating the treatment site before the treatment (either at home or in the treatment room), e.g., a warming blanket or thermal energy described herein; a modality to enable visualization during the procedure as described herein, modality to further break up fibrous septae and/or ice crystals post-treatment as described herein; and a modality to ensure symmetry post treatment, e.g., a compression band.

An exemplary method of treatment according to the invention includes creating a treatment plan, followed by pre-treatment, treatment and post-treatment.

In an aspect of the invention, a pre-treatment comprises heating, disrupting and/or preparing the treatment site immediately prior to administration of the cold solution. In some aspects, a sheath, e.g., a cannula or needle, comprising a lumen is inserted into the treatment site, and a device, e.g., an energy device, is inserted through the sheath. The energy device can be any device described herein. In some aspects, the energy device comprises one or more of an ultrasonic cutting tip, a resistive heater or a light source.

In some aspects, a sheath comprising more than one lumen (a multi-lumen sheath) is inserted into the treatment site, wherein a device, e.g., an energy device and the cold solution are administered via the more than one lumen. For example, a sheath comprising a first lumen and a second lumen, wherein a cold solution may be administered to the treatment site via the first lumen, and an energy device can be inserted into the second lumen for administration to the treatment site. Any number of working channels (or lumens) can be included, for example, two, three, four, five or six lumens, each configured to receive a device. In some aspects, a visualization device can be inserted in a lumen.

A system for the method described above comprises a sheath comprising a lumen, an energy device configured to supply energy to a subject such as a TENS device, and a cold solution supply source configured to supply a cold solution comprising liquid water and/or solid ice particles, the cold solution to be administered to a treatment site of the subject via the lumen. In another aspect, the sheath comprises a first lumen and a second lumen, wherein the energy device is configured to administer energy to the subject via the first lumen, and the cold solution is configured to be administered to the subject via the second lumen. The cold solution supply source is any suitable supply source, including but not limited to a pump system that generates flow of the cold solution through the sheath, for example, via a syringe.

In some aspects, an energy device is inserted into the treatment site through the lumen in the sheath, and directed toward the treatment site, in order to locally heat the treatment site, disrupt fascia between septae, cut tissue, provide illumination, provide imaging, collect data, and/or maximize the temperature difference experienced by the treatment site (contrast cryolipolysis) before and after administration of the cold solution. In some aspects, an energy device is administered directly into the treatment site. The energy device can comprise any of the energy devices described herein. In some aspects, the energy device is an ultrasonic device, a resistive heater, an ultrasonically actuated cutting tip, a light guide or an optical fiber.

When using a single lumen sheath, immediately prior to administration of the cold solution, the energy device may be removed from the sheath or cannula, followed by administration of the cold solution through the open lumen. In some aspects, the cold solution delivery device described above includes an ultrasound transducer which may generate heat and mechanical energy. In some aspects, generation of heat and mechanical energy will be located in a separate part of the delivery device, so as to avoid undesired heating of the cold solution. When utilizing a multi-lumen sheath, the energy device may remain in place in the first lumen, and the cold solution may be administered through the second lumen.

In some aspects, radiant energy may be administered to the treatment site through the single lumen sheath or the multi-lumen sheath, or directly, in order to preheat the treatment site prior to administering the cold solution. For example, radiant energy may be administered to the treatment site prior to administering a cold solution, e.g., a slurry or a substantially solid solution, thus maximizing the temperature difference experienced by the tissue (contrast cryolipolysis), augmenting the effects of the cold solution.

In some aspects, a cold solution is administered to the treatment site to provide cooling in order to increase the tolerability, and to limit thermal diffusion of radiant energy sources for skin treatment. For example, the cold solution may be administered to the treatment site via injection, followed by treatment of the skin at the administration site with a heat-based modality, in order to treat suprapubic fat. A cold solution delivery device may be coupled to the heating device. Such a method may enable treatment with higher levels of energy, because a cooling source is placed distal to the treatment area. A substantially solid solution may be applied on the skin to actively cool the administration site and surrounding area, followed by administration of light. The administration of the cold solution on the skin serves as a cooling source, e.g., numbing agent, to increase the tolerability of further administrations to the treatment site, e.g., laser treatment, and potentially increases the energy of traditional thermal methods of skin tightening and remodeling, including but not limited to laser, radio frequency, ultrasound skin tightening.

In an aspect of the invention, the cold solution and a secondary solution

comprising a second agent, e.g., target molecules, are simultaneously delivered to the treatment site during administration, and the second agent is activated secondarily, for example, after the original administration and/or in a home environment. For example, a multi-lumen sheath can be used, wherein the cold solution is administered via a first lumen, and the secondary solution is administered via a second lumen. A multi-lumen sheath is inserted into the treatment site, the cold solution is delivered through a first lumen, and a second agent, such as gold particles (e.g., gold microparticles or nanoparticles) in suspension, is delivered through a second lumen. At some period of time after the administration of the cold solution and the second agent, for example, after the cold solution melts, the second agent remains distributed over the area treated by the cold solution and may be externally activated, e.g., topically, to product combinatory effect. For example, light may be used to target heating of gold nanoparticles which were administered as the second agent. In some aspects, the second agent can comprise silver particles (e.g., silver microparticles or nanoparticles) or microbubble enclosed particles. In some aspects, the particles enclosed in microbubbles can be activated via an ultrasound device.

A system for the method described above comprises a sheath comprising a first lumen and a second lumen, a cold solution supply source configured to supply a cold solution comprising liquid water and/or solid ice particles, the cold solution to be administered to a treatment site of a subject via the first lumen, and a secondary solution supply source configured to supply a secondary solution comprising a second agent, the secondary solution to be administered to a treatment site of a subject via the second lumen. The second solution supply source is any suitable supply source, including but not limited to a pump system that generates flow of the second solution through the sheath, for example via a syringe.

In an aspect of the invention, a cold solution is administered to a treatment site, followed by use of an additional modality, e.g., a laser, to topically heat the treatment site. The additional modality may be selectively applied, such that the cold solution is melted in selected areas. In some aspects, the melted areas may result in decreased therapeutic effect. In some aspects, the use of selective melting may provide a desired contouring effect in or around the treatment site.

In an aspect of the invention, a pre-treatment comprises heating, disrupting and/or preparing the treatment site immediately prior to administration of the cold solution, wherein the pre-treatment utilizes a fenestrated needle. In one aspect, the fenestrated needle is inserted into the treatment site, followed by injection of a target molecule solution, such as gold particles, silver particles, microbubble enclosed particles, or another chemical component, into the treatment site. The target molecule solution is supplied by a target molecule solution supply source, which is any suitable supply source, including but not limited to a pump system that generates flow of the target molecule solution through the fenestrated needle. After injection of the target molecule solution, the fenestrated needle is removed. In another aspect, after injection of the treatment site, energy is administered through the lumen of the fenestrated needle. The energy source may be an ultrasonic device, a resistive heater, an ultrasonically actuated cutting tip, a light guide or an optical fiber, which may locally heat the treatment site, disrupt fascia between septae, cut tissue, provide illumination, provide imaging, collect data, and/or maximize the temperature difference experienced by the treatment site (contrast cryolipolysis). For example, a target molecule solution can be light activated through the fenestrations of a fenestrated needle. Prior to administration of the cold solution, the energy device can be removed. In one aspect, the cold solution is administered via a delivery device attached to the fenestrated needle. In another aspect, the fenestrated needle is withdrawn, and the cold solution is delivered via a delivery device.

In some aspects, pre-treatment includes obtaining measurements of the treatment site and/or the surrounding area through imaging, including but not limited to Magnetic Resonance Imaging (MRI), Computed Topography (CT), ultrasound, Positron Emission Tomography (PET), 3D imaging, and combinations thereof. Imaging may be used in the absence of, or together with, an incision near or at the treatment site. In another aspect, measurements of the treatment site and/or the surrounding area may be obtained through a computer or artificial intelligence system, which contains data obtained from the subject to be treated and/or data obtained from multiple subjects.

In some aspects, pre-treatment also include a heat application at or near the injection site, which may improve cryolipolysis due to the difference in temperature between the warmed injection site and the cold solution to be administered, i.e., contrast cryolipolysis.

In some aspects, pre-treatment also includes selective disruption of fibrous tissue at the treatment and/or injection site and the surrounding area, thus enabling a smoothing effect in addition to the fat reduction. The selective disruption of fibrous tissue may occur through any suitable method, including but not limited to, mechanical vibration, application of heat, and/or topical or subcutaneous administration of energy activated nanoparticles, e.g., gold or silver. The selective disruption of fibrous tissue may also occur by administration of a cold solution, which may be utilized to mechanically disrupt fibrous tissue to break up compartments found within the subcutaneous fat, allowing the subcutaneous fat to spread and create a visually smoother appearance, for example in the treatment of cellulite, as described in U.S. Provisional Application No. 62/953,272, previously incorporated by reference, and in International Patent Application Ser. No. PCT/US2017/059947 filed on Nov. 13, 2017, the entirety of which is incorporated herein by reference.

In some aspects, pre-treatment also includes fractional wounding by drilling holes at or near the treatment site, in order to disrupt fibrous tissue and/or stimulate collagen production.

In some aspects, treatment includes injection of a cold solution to the subject, in any suitable amount, in the methods described above, optionally together with one or more pharmacological agents, as described above, or energy activated nanoparticles, such as gold or silver. The cold solution, one or more pharmacological agents, and/or energy activated nanoparticles may be administered simultaneously and/or separately. If the cold solution, one or more pharmacological agents and/or energy activated nanoparticles are administered separately, the cold solution may be administered before, after, or before and after the administration of the one or more pharmacological agents and/or energy activated nanoparticles.

In some aspects, treatment includes heat application in order to improve cryolipolysis, application of mechanical energy, such as vibration, massage, pulsation, and/or compression, to assist with cell death after cold solution is injected, fractional wounding to deliver cold solution, and/or administration of resorbable sutures.

In some aspects, post-treatment includes one or more of magnetic muscle stimulation (MMS) in order to develop/improve muscle tone, compression, thermal compression, cool/cold compression, activation of previously deposited energy activated nanoparticles, nutrition planning and monitoring, exercise, topical application of a microneedle patch to allow transdermal delivery of one or more pharmacological agents, as described above, and/or the collection of samples, including but not limited to blood and interstitial fluid, which may utilized for data collection and analysis, collection of data using a computer program or application, and/or fractional wounding in order to stimulate collagen production.

All documents, books, manuals, patents, published patent applications, and other reference materials cited herein are incorporated by reference in their entirety.

While the invention has been described with reference to certain particular aspects thereof, those skilled in the art will appreciate that various modifications may be made without departing from the spirit and scope of the invention. The scope of the appended claims is not to be limited to the specific embodiments described.

What is claimed is:

1. A method for treating suprapubic fat, said method comprising administering an effective amount of a cold solution to a treatment site of a subject, wherein the treatment site comprises suprapubic fat, and wherein the cold solution comprises liquid water and/or solid ice particles.

2. The method for treatment of claim 1, wherein the cold solution comprises about 2% to about 70% solid ice particles, and optionally one or more additives.

3. The method for treatment of claim 1, wherein the cold solution comprises about 71% to about 100% solid ice particles, and optionally one or more additives.

4. The method for treatment of claim 1, wherein the cold solution is substantially liquid, and optionally comprises one or more additives.

5. The method for treatment of claim 1, wherein prior to administering an effective amount of the cold solution, imaging the treatment site of the subject to identify the suprapubic region.

6. The method for treatment of claim 1, wherein prior to administering an effective amount of the cold solution, physically and/or thermally isolating the treatment site.

7. The method for treatment of claim 1, wherein the cold solution is administered in a single treatment, or in a series of treatments.

8. The method for treatment according to claim 1, wherein the cold solution is administered via a device selected from the group consisting of a needle, an expandable needle, a needle comprising more than one tip, a fenestrated needle, a fenestrated cannula, and an implant.

9. A method for penile enhancement, said method comprising administering an effective amount of a cold solution to a treatment site of a subject in need thereof, wherein the treatment site comprises suprapubic fat, wherein the cold solution comprises liquid water and/or solid ice particles, and optionally one or more additives, and wherein the method results in a reduction in suprapubic fat.

10. The method for treatment of claim 9, wherein the cold solution comprises about 2% to about 70% solid ice particles, and optionally one or more additives.

11. The method for treatment of claim 9, wherein the cold solution comprises about 71% to about 100% solid ice particles, and optionally one or more additives.

12. The method for treatment of claim 9, wherein the cold solution is substantially liquid, and optionally comprises one or more additives.

13. The method for treatment of claim 9, wherein prior to administering an effective amount of a cold solution, imaging the treatment site of the subject to determine the presence of suprapubic fat.

14. The method for treatment of claim 9, wherein prior to administering an effective amount of the cold solution, physically and/or thermally isolating the treatment site.

15. The method for treatment of claim 9, wherein the cold solution is administered in a single treatment, or in a series of treatments.

16. The method for treatment according to claim 9, wherein the cold solution is administered via a device selected from the group consisting of a needle, an expandable needle, a needle comprising more than one tip, a fenestrated needle, a fenestrated cannula, and an implant.

17. A method for treating a medical condition, said method comprising administering an effective amount of a cold solution to a treatment site of a subject in need thereof, wherein the treatment site comprises suprapubic fat, wherein the cold solution comprises liquid water and/or solid ice particles, and optionally one or more additives, and wherein the method results in a reduction in suprapubic fat.

18. The method for treatment of claim 17, wherein the cold solution comprises about 2% to about 70% solid ice particles, and optionally one or more additives.

19. The method for treatment of claim 17, wherein the cold solution comprises about 71% to about 100% solid ice particles, and optionally one or more additives.

20. The method for treatment of claim 17, wherein the cold solution is substantially liquid, and optionally comprises one or more additives.

21. The method for treatment of claim 17, wherein prior to administering an effective amount of a cold solution, imaging the treatment site of the subject to determine the presence of suprapubic fat.

22. The method for treatment of claim 17, wherein prior to administering an effective amount of the cold solution, physically and/or thermally isolating the treatment site.

23. The method for treatment of claim 17, wherein the cold solution is administered in a single treatment, or in a series of treatments.

24. The method for treatment according to claim 17, wherein the cold solution is administered via a device selected from the group consisting of a needle, an expandable needle, a needle comprising more than one tip, a fenestrated needle, a fenestrated cannula, and an implant.

25. A composition comprising a cold solution, wherein:
the cold solution (1) comprises about 2% to about 70% solid ice particles, (2) comprises about 71% to about 100% solid ice particles, or (3) is substantially liquid;
the composition optionally comprises at least one additive; and optionally one or more suitable pharmacological agents; and
the composition is used to treat suprapubic fat in a subject.

26. A method for treating suprapubic fat in a subject, said method comprising:
imaging one or more potential treatment sites in the subject to determine the presence of suprapubic fat,
thermally and/or physically isolating one or more potential treatment sites,
selecting one or more treatment sites which contain suprapubic fat, and
administering a cold solution into to the one or more selected treatment sites to treat the suprapubic fat of the subject,
wherein the cold solution comprises liquid water and/or ice particles, optionally one or more additives, and optionally one or more suitable pharmacological agents.

27. A system for treating suprapubic fat, the system comprising:
a delivery device for delivering a cold solution comprising liquid water and/or ice particles, optionally one or more additives, and optionally one or more suitable pharmacological agents.

28. The system of claim 27, further comprising an imaging device.

29. The system of claim 27, further comprising an isolation device.

30. The system of claim 27, wherein the isolation device comprises a ring.

31. The system of claim 27, wherein the isolation device comprises a sleeve.
